(19) United States
(12) Patent Application Publication
(10) Pub. No.: US 2025/0268222 A1
(43) Pub. Date: Aug. 28, 2025

(54) COTTON VARIETY FM 823AXTP

(71) Applicant: BASF Agricultural Solutions US LLC, Research Triangle Park, NC (US)

(72) Inventors: Anna J. Hall, Lubbock, TX (US); Cory Mills, Lubbock, TX (US)

(73) Assignee: BASF Agricultural Solutions US LLC, Research Triangle Park, NC (US)

(21) Appl. No.: 19/064,067

(22) Filed: Feb. 26, 2025

Related U.S. Application Data
(60) Provisional application No. 63/558,154, filed on Feb. 27, 2024.

(51) Int. Cl. A01H 6/60; A01H 5/10

(52) U.S. Cl. CPC A01H 6/604; A01H 5/10

(57) ABSTRACT

A cotton variety FM 823AXTP is disclosed. Seeds, plants, plant cells, plant tissue, harvested products and cotton lint as well as to hybrid cotton plants and seeds obtained by repeatedly crossing plants of variety FM 823AXTP with other plants are also provided. Plants and varieties produced by the method of essential derivation from plants of FM 823AXTP and to plants of FM 823AXTP reproduced by vegetative methods, including but not limited to tissue culture of regenerable cells or tissue from FM 823AXTP are provided.

### FIELD OF THE DISCLOSURE

This disclosure relates to the field of plant breeding. More particularly, one or more embodiments relate to a variety of cotton designated as FM 823AXTP, its essentially derived varieties and the hybrid varieties obtained by crossing FM 823AXTP as a parent line with plants of other varieties or parent lines.

### BACKGROUND OF THE DISCLOSURE

Cotton is an important, fiber producing crop. Due to the importance of cotton to the textile industry, cotton breeders are increasingly seeking to obtain healthy, good yielding crops of excellent quality.

Cotton is commonly reproduced by self-pollination and fertilization. This type of sexual reproduction facilitates the preservation of plant and variety characteristics during breeding and seed production. The preservation of these characteristics is often important to plant breeders for producing cotton plants having desired traits. By carefully choosing the breeding parents, the breeding and selection methods, the testing layout and testing locations, a cotton breeder may breed a particular cotton variety type.

### SUMMARY OF VARIOUS ASPECTS OF THE DISCLOSURE

Seeds of the cotton variety FM 823AXTP are provided. Also, provided are seeds, plants, plant cells, parts of plants, cotton lint or fiber, and cotton textiles of cotton variety FM 823AXTP as well as to hybrid cotton plants and seeds obtained by repeatedly crossing plants of FM 823 AXTP with other cotton plants, and Essentially Derived Varieties of cotton variety FM 823 AXTP. The disclosure encompasses plants and plant varieties produced by the method of derivation or essential derivation from plants of FM 823 AXTP and to plants of FM 823 AXTP reproduced by vegetative methods, including but not limited to regeneration of embryogenic cells or tissue of FM 823AXTP. The disclosure also encompasses methods of producing cotton seeds that comprise crossing plants of cotton variety FM 823AXTP either with itself or with a second, distinct cotton plant.

### DETAILED DESCRIPTION OF VARIOUS ASPECTS OF THE DISCLOSURE

The present disclosure relates to a new and distinctive cotton variety FM 823AXTP, its seeds, plants, plant parts and hybrids thereof. The cotton variety of the present disclosure has been obtained by a general breeding process comprising the steps outlined below. For reference, see chapter 11, “Breeding Self-Pollinated Crops by Hybridization and Pedigree Selection” in Briggs and Knowles (1967).

Parent plants, which have been selected for good agronomic and fiber quality traits are manually crossed in different combinations. The resulting F₁ (Filial generation 1) plants are self-fertilized and the resulting F₂ generation plants are planted in a selection field.

These F₂ plants are observed during the growing season for health, growth vigor, plant type, plant structure, leaf type, stand ability, flowering, maturity, seed yield, boll type, boll distribution, boll size, fiber yield and fiber quality. Plants are then selected based on desired characteristics. The selected plants are harvested, and the bolls analyzed for fiber characteristics. The harvested seeds are cleaned and stored. This procedure is repeated in the following growing seasons, whereby the selection and testing units increase from individual plants in the F₂ generation, to multiple plant containing ‘lines’ (descending from one mother plant) in the F₅ generation. The number of units decrease from approximately 2500 plants in the F₂ generation to 20 lines in the F₅ generation by selecting about 10-20% of the units in each selection cycle.

The increased size of the units, whereby more seed per unit is available, allows the selection and testing in replicated trials on more than one location with a different environment and a more extensive and accurate analysis of the fiber quality.

The lines or candidate varieties become genotypically more homozygous and phenotypically more homogeneous by selecting similar plant types within a line and by discarding the so called off-types from the very variable F₂ generation on to the final F₇ or F₈ generation.

The plant breeder may monitor the results through each filial generation, and depending on the intermediate results, may decide to vary the procedure described above, such as by accelerating the process by testing a particular line earlier or retesting a line another year. The breeder may also select plants for further crossing with existing parent plants or with other plants resulting from the current selection procedure.

By the method of recurrent backcrossing, as described by Briggs and Knowles, supra, in chapter 13, “The Backcross Method of Breeding”, the breeder may introduce a specific trait or traits into an existing valuable line or variety, while otherwise preserving the unique combination of characteristics of this line or variety. In this crossing method, the valuable parent is recurrently used to cross it at least two or three times with each resulting backcross F₁, followed by selection of the recurrent parent plant type, until the phenotype of the resulting F₁ is similar or almost identical to the phenotype of the recurrent parent with the addition of the expression of the desired trait or traits.

This method of recurrent backcrossing eventually results in an essentially derived variety, which is predominantly derived from the recurrent parent or initial variety. This method can therefore also be used to get as close as possible to the genetic composition of an existing successful variety. Thus, compared to the recurrent parent, the essentially derived variety retains a distinctive trait, which can be any phenotypic trait, with the intention to profit from the qualities of that successful initial variety.

Depending on the number of backcrosses and the efficacy of the selection of the recurrent parent plant type and genotype, which can be supported by the use of molecular markers (See P. Stam, “Marker-assisted introgression: speed at any cost?” Proceedings of the Eucarpia Meeting on Leafy Vegetable Genetics and Breeding, Noordwijkerhout, The Netherlands, 19-21 (March 2003). Eds. Th. J. L. van Hintum, A. Lebeda, D. Pink, J. W. Schut. P117-124), the genetic conformity with the initial variety of the resulting essentially derived variety may vary between 90% and 100%.

The relatedness can, for example be determined by fingerprinting techniques (e.g., making use of isozyme markers and/or molecular markers such as SNP markers, AFLP markers, microsatellites, minisatellites, RAPD markers, RFLP markers and others). A plant is “closely related” to FM 823AXTP if its DNA fingerprint is at least 80%, 90%, 95% or 98% identical to the fingerprint of FM 823AXTP. In one embodiment, AFLP markers are used for DNA fingerprinting (Vos et al. 1995, Nucleic Acid Research 23:4407-4414). A closely related plant may have a Jaccard's Similarity index of at least about 0.8, preferably at least about 0.9, 0.95, 0.98 or more (Pisanu et al. ISHS 2004, Acta Hort. 660).

Other than recurrent backcrossing, as described herein, such essentially derived variety may also be obtained by the selection from an initial variety of an induced or natural occurring mutant plant, or of an occurring variant (off-type) plant, or of a somaclonal variant plant, or by genetic transformation of regenerable plant tissue or embryogenic cell cultures of the said initial variety by methods well known to those skilled in the art, such as Agrobacterium-mediated transformation as described by H. F. Sakhanoko et al 2004: “Induction of Somatic embryogenesis and Plant Regeneration in Select Georgia and Pee Dee Cotton Lines”, Crop Science 44:2199-2205, Reynaerts et al 2000: “Improved method for Agrobacterium mediated transformation of cotton”, Patent application number WO 0071733, Umbeck et al 1988: “Genetic engineering of cotton plants and lines”, Patent application number EP0290355 and others. Examples of transgenic events transformed in this way are “LLCotton25,” USDA-APHIS petition 02-042-01p, “Cot 102,” USDA-APHIS petition 03-155-01p, and “281-24-236,” USDA-APHIS petition 03-036-01p combined with “3006-210-23,” USDA-APHIS petition 03-036-02p. Information regarding these and other transgenic events referred to herein may be found at the U.S. Department of Agriculture's (USDA) Animal and Plant Health Inspection Service (APHIS) website. An “Event” is defined as a (artificial) genetic locus that, as a result of genetic engineering, carries a foreign DNA comprising at least one copy of the gene(s) of interest. Other methods of genetic transformation are well known in the art such as microprojectile bombardment. See, e.g., U.S. Publication No. 20090049564, which is incorporated by reference herein in its entirety.

The plants selected or transformed retain the unique combination of the characteristics of FM 823AXTP, except for the characteristics (e.g., one, two, three, four or five characteristics) changed by the selection of the mutant or variant plant or by the addition of a desired trait via genetic transformation. Therefore, the product of essential derivation (i.e., an essentially derived variety), has the phenotypic characteristics of the initial variety, except for the characteristics that change as a result of the act of derivation. Plants of the essentially derived variety can be used to repeat the process of essential derivation. The result of this process is also a variety essentially derived from said initial variety.

In one embodiment, FM 823AXTP progeny plants are produced by crossing plants of FM 823 AXTP with other, different or distinct cotton plants, and further selfing or crossing these progeny plants with other, distinct plants and subsequent selection of derived progeny plants. The process of crossing FM 823AXTP derived progeny plants with itself or other distinct cotton plants and the subsequent selection in the resulting progenies can be repeated up to 7 or 8 times in order to produce FM 823AXTP derived cotton plants.

Another embodiment provides for seeds, plants, plant cells and parts of plants of the cotton variety FM 823AXTP.

In one embodiment, plants produced by growing such seeds are provided. Also provided herein are pollen or ovules of these plants, as well as a cell or tissue culture of regenerable cells from such plants. In another embodiment, a cotton plant regenerated from such cell or tissue culture, wherein the regenerated plant has the morphological and physiological characteristics of cotton cultivar FM 823AXTP when grown in the same environmental conditions is provided. In yet another embodiment, methods of testing for a plant having the morphological and physiological characteristics of cotton cultivar FM 823AXTP is provided. In one embodiment, the testing for a plant having the morphological and physiological characteristics of cotton cultivar FM 823 AXTP is performed in the same field, under the same conditions and in the presence of plants of FM 823AXTP, e.g., plants grown from the representative seed deposited with a Budapest Treaty depository. In another embodiment, the characteristics to be tested are those described herein.

In another embodiment, regenerable cells for use in tissue culture of cotton cultivar FM 823 AXTP are provided. The tissue culture will preferably be capable of regenerating plants having the physiological and morphological characteristics of the cotton cultivar FM 823AXTP as described herein (e.g., Tables 1-3), and of regenerating plants having substantially the same genotype as the cotton plant described herein. In one embodiment, the regenerable cells in such tissue cultures will be from embryos, protoplasts, meristematic cells, callus, pollen, leaves, anthers, pistils, roots, root tips, flowers, seeds, pods or stems. Still further, cotton plants regenerated from the tissue cultures of the embodiments are provided.

In yet another embodiment, a cotton plant of the cotton variety FM 823 AXTP comprising at least a first transgene, wherein the cotton plant is otherwise capable of expressing all the physiological and morphological characteristics of the cotton variety FM 823AXTP as described herein (e.g., Tables 1-3) is provided. In another embodiment, a plant is provided that comprises a single locus conversion. A single locus conversion may comprise a transgenic gene which has been introduced by genetic transformation into the cotton variety FM 823AXTP or a progenitor thereof. A transgenic or non-transgenic single locus conversion can also be introduced by backcrossing, as is well known in the art. In certain embodiments, the single locus conversion may comprise a dominant or recessive allele. The locus conversion may confer potentially any desired trait upon the plant as described herein.

Single locus conversions may be implemented by a plant breeding technique called backcrossing wherein essentially all of the desired morphological and physiological characteristics of a variety are recovered in addition to the characteristics conferred by the single locus transferred into the variety via the backcrossing technique. A single locus may comprise one gene, or in the case of transgenic plants, one or more transgenes integrated into the host genome at a single site (locus).

Another embodiment provides for a method of introducing a single locus conversion into cotton cultivar FM 823AXTP comprising: (a) crossing the FM 823AXTP plants, grown from seed deposited with a Budapest Treaty depository, with plants of another cotton line that comprise a desired single locus to produce F₁ progeny plants; (b) selecting F₁ progeny plants that have the single locus to produce selected F₁ progeny plants; (c) crossing the selected F₁ progeny plants with the FM 823 AXTP plants to produce first backcross progeny plants; (d) selecting for first backcross progeny plants that have the desired single locus and the physiological and morphological characteristics of cotton cultivar FM 823AXTP as described herein (e.g., Tables 1-3), when grown in the same environmental conditions, to produce selected first backcross progeny plants; and (e) repeating steps (c) and (d) one or more times (e.g. one, two, three, four, etc. times) in succession to produce selected second or higher backcross progeny plants that comprise the desired single locus and all of the physiological and morphological characteristics of cotton cultivar FM 823AXTP as described herein (e.g., Tables 1-3), when grown in the same environmental conditions is provided. Plants produced by this method have all of the physiological and morphological characteristics of FM 823AXTP, except for the characteristics derived from the desired trait.

In another embodiment, a method of producing an essentially derived plant of cotton variety FM 823AXTP comprising introducing a transgene conferring the desired trait into the plant, resulting in a plant with the desired trait and all of the physiological and morphological characteristics of cotton variety FM 823AXTP when grown in the same environmental conditions is provided. In another embodiment, a method of producing an essentially derived cotton plant from FM 823AXTP comprising genetically transforming a desired trait in regenerable cell or tissue culture from a FM 823 AXTP, resulting in an essentially derived cotton plant that retains the expression of the phenotypic characteristics of cotton variety FM 823 AXTP, except for the characteristics changed by the introduction of the desired trait.

Desired traits described herein include modified cotton fiber characteristics, herbicide resistance, insect or pest resistance, disease resistance, including bacterial or fungal disease resistance, male sterility, modified carbohydrate metabolism and modified fatty acid metabolism. Such traits and genes conferring such traits are known in the art. See, e.g., US20090049564, incorporated by reference herein in its entirety.

In another embodiment, methods wherein the desired trait is herbicide tolerance, and the tolerance is linked to an herbicide such as glyphosate, glufosinate, sulfonylurea, dicamba, phenoxy proprionic acid, cyclohexanedione, triazine, benzonitrile, bromoxynil or imidazalinone are provided.

In one embodiment, the desired trait is insect resistance conferred by a transgene encoding a Bacillus thuringiensis (Bt) endotoxin, a derivative thereof, or a synthetic polypeptide modeled thereon.

Also included herein is a method of producing cotton seed, comprising the steps of using the plant grown from seed of cotton variety FM 823AXTP, of which a representative seed sample will be deposited with a Budapest Treaty depository, as a recurrent parent in crosses with other cotton plants different from FM 823AXTP, and harvesting the resultant cotton seed.

In another embodiment, seeds, plants, plant cells and parts of plants of cotton varieties that are essentially derived from FM 823AXTP, being essentially the same as FM 823AXTP by expressing the unique combination of characteristics of FM 823 AXTP, except for the agronomic characteristics (e.g., one, two, three, four, or five, characteristics) being different from the characteristics of FM 823 AXTP as a result of the act of derivation.

Another embodiment provides for the reproduction of plants of FM 823AXTP by the method of tissue culture from any regenerable plant tissue obtained from plants of variety FM 823 AXTP. Plants reproduced by this method express the specific combination of characteristics of variety FM 823AXTP and fall within its scope. During one of the steps of the reproduction process via tissue culture, somaclonal variant plants may occur. These plants can be selected as being distinct, but still fall within the scope of this disclosure as being essentially derived from this disclosure.

Another embodiment provides for a method of producing an inbred cotton plant derived from the cotton variety FM 823AXTP comprising: (a) preparing a progeny plant derived from cotton variety FM 823 AXTP, a representative sample of seed of said variety deposited with a Budapest Treaty depository, by crossing cotton variety FM 823AXTP with a cotton plant of a second variety; (b) crossing the progeny plant with itself or a second plant to produce a seed of a progeny plant of a subsequent generation; (c) growing a progeny plant of a subsequent generation from said seed and crossing the progeny plant of a subsequent generation with itself or a second plant; and (d) repeating steps (b) and (c) for an additional 3-10 generations with sufficient inbreeding to produce an inbred cotton plant derived from the cotton variety FM 823 AXTP.

Another embodiment of this disclosure is the production of a hybrid variety, comprising repeatedly crossing plants of FM 823AXTP with plants of a different variety or varieties or with plants of a non-released line or lines. In practice, three different types of hybrid varieties may be produced (see e.g., Chapter 18, “Hybrid Varieties” in Briggs and Knowles, supra).

The “single-cross hybrid” produced by two different lines, the “three-way hybrid”, produced by three different lines such that first the single hybrid is produced by using two out of the three lines followed by crossing this single hybrid with the third line, and the “four-way hybrid” produced by four different lines such that first two single hybrids are produced using the lines two by two, followed by crossing the two single hybrids so produced.

Each single, three-way or four-way hybrid variety so produced and using FM 823 AXTP as one of the parent lines contains an essential contribution of FM 823AXTP to the resulting hybrid variety and falls within the scope of this disclosure.

In one embodiment, cotton lint or fiber produced by the variety FM 823AXTP, plants reproduced from the variety FM 823AXTP, and plants essentially derived from the variety FM 823 AXTP. The final textile produced from the unique fiber of FM 823AXTP also falls within the scope of this disclosure. A method of producing a commodity plant product (e.g., lint, cotton seed oil) comprising obtaining a plant of the present disclosure or a part thereof, and producing said commodity plant product therefrom is also within the scope of this disclosure.

The entire disclosure of each document cited herein (e.g., US patent publications, non-patent literature, etc.) is hereby incorporated by reference.

### Variety Description

FM 823AXTP was derived from intercrossing of multiple backcross populations, all containing 112TX0248 background which has Bacterial Leaf Blight (BLB) resistance, in the Memphis, TN greenhouses. The 112TX0248 GLITP BC4F1 population was made as follows: in 2016, the initial F1 cross was made between 112TX0248 and the donor parent 1060323 IFT+TLP @F1 contributing the BASF AXANT, GLYTOL®, LIBERTYLINK®, TWINLINK® Plus traits; 4 further backcrosses from 2016-2017 were made to 112TX0248 to generate 112TX0248 GLITP BC4F1 seeds. The 112TX0248 RKN1 BC4F1 population was made as follows: in 2016, the initial F1 cross was made between 112TX0248 and the donor parent 13Q2UGL-0707 contributing the Root Knot Nematode (RKN1) resistance trait; 4 further backcrosses from 2016-2017 were made to 112TX0248 to generate 112TX0248 RKN1 BC4F₁ seeds. The 112TX0248 XTL BC4F1 population was made as follows: in 2017 Q1, the initial F₁ cross was made between 112TX0248 and the donor parent 08T0026C GLTD @BC2F₂ contributing the Bayer XTENDLINK™ trait and the BASF LIBERTYLINK®, TWINLINK® traits; 4 further backcrosses from 2017-2018 were made to 112TX0248 to generate 112TX0248 XTL BC4F1 seeds. In 2018 Q1, 112TX0248 RKN1 BC4F1 plants were used as the male parent and crossed with 112TX0248 GLITP BC4F1 plants as the female parent to generate 112TX0248 GLITP+RKN1 F1 seeds. In 2019 Q1, 112TX0248 GLITP+RKN1 F1 plants were used as the male parent and crossed with 112TX0248 XTL BC4F1 plants as the female parent to generate 112TX0248 GLIXTP+RKN1 F1 seeds. In 2019 Q2, 112TX0248 GLIXTP+RKN1 F1 plants were generated and tested for GLIXTP (MON88701, GHB811, T304-40, GHB119, COT102), RKN1 and BLB. Plants found homozygous, hemizygous, or heterozygous for these traits were grown out in Memphis, TN greenhouses and F2 seeds were harvested. In 2020 Q1, individual F2 plants found homozygous for GLIXTP, RKN1, BLB and Bronze Wilt Resistance (BWR) were selected as progeny plants and IPS harvested. In 2020-2021, F3 progeny rows were grown for seed increase in Guanica, PR.

Table 1 shows the characteristics of cotton variety FM 823AXTP. Table 2 shows the disease responses of cotton variety FM 823AXTP, Table 3 shows the nematode/insect/pest responses to cotton variety FM 823AXTP, and Table 4 shows a comparison of yield and fiber data between FM 823 AXTP and FM 2398GLTP collected from 5 field locations surrounding Lubbock, Texas areas in 2023.

TABLE 1
Variety Description and Characteristics of FM 823AXTP

| Description of characteristic | Possible expression/note | FM 823AXTP |
|---|---|---|
| GENERAL PLANT TYPE | | |
| Species | | Gossypium hirsutum L. |
| Plant Habit | spreading, intermediate, compact | Compact |
| Foliage | sparse, intermediate, dense | Intermediate |
| Stem Lodging | lodging, intermediate, erect | Erect |
| Fruiting Branch | clustered, short, normal | Normal |
| Growth | determinate, intermediate, | Intermediate |
| | indeterminate | |
| Leaf color | greenish yellow, light green, medium | Medium Green |
| | green, dark green | |
| Boll Shape | Length < Width, L = W, L > W | L > W |
| Boll Breadth | broadest at base, broadest at middle | Broadest at Middle |
| MATURITY | | |
| | Visual rating of percent open bolls | 62.8 |
| | when all testing lines in the test were | |
| | about 40% open | |
| PLANT | | |
| cm. to first Fruiting Branch | from cotyledonary node | 16.1 |
| No. of nodes to 1st Fruiting | excluding cotyledonary node | 7 |
| Branch | | |
| Mature Plant Height in cm. | cotyledonary node to terminal | 55.6 |
| LEAF: Upper most, fully | | |
| expanded leaf | | |
| Type | normal, sub-okra, okra, super-okra | Normal |
| Pubescence | absent, sparse, medium, dense | Sparse |
| Nectaries | present, absent | Present |
| Stem Pubescence | | |
| | glabrous, intermediate, hairy | Intermediate |
| GLANDS (Gossypol) | | |
| | absent, sparse, normal, more than | |
| | normal | |
| Leaf | | Normal |
| Stem | | Normal |
| Calyx lobe | (normal, normal is absent) | Normal |
| FLOWER | | |
| Petals color | cream, yellow | Cream |
| Pollen color | cream, yellow | Cream |
| Petal Spot | present, absent | Absent |
| SEED | | |
| Seed Index | g/100 seed fuzzy basis | 10.8 |
| BOLL | | |
| Lint percent, picked | | 0.42 |
| Number of Seeds per Boll | | 26.8 |
| Grams Seed Cotton per Boll | | 5.55 |
| Number of Locules per Boll | | 4.5 |
| Boll Type | storm proof, storm resistant, open | Storm Resistant |
| FIBER PROPERTIES | | |
| Length, inches, 2.5% SL | | 1.13 |
| Uniformity (%) | | 83.2 |
| Strength, T1 (g/tex) | | 30.1 |
| Elongation, E1 (%) | | 6.8 |
| Micronaire | | 4.27 |

TABLE 2
DISEASES

| Disease | FM 823AXTP |
|---|---|
| Bacterial Blight Race 18 | Resistant |
| Verticillium Wilt | Moderately Resistant |

TABLE 3
NEMATODES, INSECTS, AND PESTS

| Nematode/Insect/Pest | FM 823AXTP |
|---|---|
| Root Knot Nematode | Moderately Resistant |
| Boll Worm | Resistant |
| Pink Bollworm | Resistant |
| Fall Armyworm | Moderately Resistant |
| Tobacco Bud Worm | Resistant |

TABLE 4
Yield and fiber data collected from 5 field locations

| | | | | | Boll | | | | | |
|---|---|---|---|---|---|---|---|---|---|---|
| Material Name | Yield | GTO | Lint % | Visual | Type | Leng | Stren | Mic | UR | Elong |
| FM 823AXTP | 1058 | 0.337 | 0.42 | 6.7 | 5.4 | 1.13 | 30.1 | 4.27 | 83.2 | 6.8 |
| FM 2398GLTP | 1101 | 0.336 | 0.41 | 6.5 | 4.1 | 1.16 | 29.9 | 4.85 | 83.2 | 6.3 |
| Mn | 1079 | 0.346 | 0.42 | 6.5 | 3.8 | 1.14 | 30.1 | 4.53 | 82.4 | 6.4 |
| LSD(.05) | 102 | 0.021 | 0.01 | 0.7 | 0.5 | 0.02 | 1.2 | 0.24 | 0.8 | 0.2 |
| CVErr | 6.59 | 2.40 | 1.18 | 6.93 | 8.27 | 1.22 | 1.94 | 1.88 | 0.54 | 1.97 |
| SigEnt | 0.00 | 0.00 | 0.00 | 0.00 | 0.00 | 0.00 | 0.00 | 0.00 | 0.00 | 0.00 |
| #Locs | 5 | 2 | 5 | 4 | 4 | 5 | 5 | 5 | 5 | 5 |
| Total Reps | 12 | 3 | 12 | 7 | 7 | 8 | 8 | 8 | 8 | 8 |

### Definitions

In the description and tables which follow, a number of terms are used. In order to provide a clear and consistent understanding of the specification and claims, the following definitions are provided:

A: When used in conjunction with the word “comprising” or other open language in the claims, the words “a” and “an” denote “one or more.

Allele: Any of one or more alternative forms of a gene locus, all of which alleles relate to one trait or characteristic. In a diploid cell or organism, the two alleles of a given gene occupy corresponding loci on a pair of homologous chromosomes.

Backcrossing: A process in which a breeder repeatedly crosses hybrid progeny, for example a first-generation hybrid (F₁), back to one of the parents of the hybrid progeny. Backcrossing can be used to introduce one or more single locus conversions from one genetic background into another.

Cm to FFB: Measure of centimeters to first fruiting branch.

COT102: the man-made cotton locus containing the vip3A gene and further described in APHIS petition No. 03-155-01p.

Crossing: The mating of two parent plants.

Cross-pollination: Fertilization by the union of two gametes from different plants.

Desired Agronomic Characteristics: Agronomic characteristics (which will vary from crop to crop and plant to plant) such as yield, maturity, pest resistance and lint percent which are desired in a commercially acceptable crop or plant. For example, improved agronomic characteristics for cotton include yield, maturity, fiber content and fiber qualities.

Diploid: A cell or organism having two sets of chromosomes.

Disease Resistance: The ability of plants to restrict the activities of a specified pest, such as an insect, fungus, virus, or bacterial.

Disease Tolerance: The ability of plants to endure a specified pest (such as an insect, fungus, virus or bacteria) or an adverse environmental condition and still perform and produce in spite of this disorder.

Donor Parent: The parent of a variety which contains the gene or trait of interest which is desired to be introduced into a second variety.

E1: Refers to elongation, a measure of fiber elasticity (high=more elastic).

Emasculate: The removal of plant male sex organs or the inactivation of the organs with a cytoplasmic or nuclear genetic factor conferring male sterility or a chemical agent.

Essentially all the physiological and morphological characteristics: A plant having essentially all the physiological and morphological characteristics means a plant having the physiological and morphological characteristics, except for the characteristics derived from the desired trait.

F₁ Hybrid: The first-generation progeny of the cross of two nonisogenic plants.

Fallout (Fo): As used herein, the term “fallout” refers to the rating of how much cotton has fallen on the ground at harvest.

FB5 cm to FFN: Measure of centimeters from main stem to first fruiting node at fruiting branch 5.

2.5% Fiber Span Length: Refers to the longest 2.5% of a bundle of fibers expressed in inches as measured by a digital fibergraph.

Fiber Characteristics: Refers to fiber qualities such as strength, fiber length, micronaire, fiber elongation, uniformity of fiber and amount of fiber.

Fiber Elongation: Sometimes referred to as E1, refers to the elongation of the fiber at the point of breakage in the strength determination as measured by High Volume Instrumentation (HVI).

Fiber Span Length: The distance spanned by a specific percentage of fibers in a test specimen, where the initial starting point of the scanning in the test is considered 100 percent as measured by a digital fibergraph.

Fiber Strength (Str): Denotes the force required to break a bundle of fibers. Fiber strength is expressed in grams per tex on an HVI.

Fruiting Nodes: The number of nodes on the main stem from which arise branches that bear fruit or boll in the first position.

Genotype: The genetic constitution of a cell or organism.

Gin Turnout: Refers to fraction of lint in a machine harvested sample of seed cotton (lint, seed, and trash).

GLT: a breeding stack of GlyTo1 together with Twinlink.

Glyto1: the man-made cotton locus containing the 2mepsps gene and further described in APHIS petition No. 05-217-05n (referred to as GHB614).

GLTP: a breeding stack of GlyTo1 together with Twinlink and COT102.

Haploid: A cell or organism having one set of the two sets of chromosomes in a diploid.

Length (Len): The fiber length in inches using an HVI.

Linkage: A phenomenon wherein alleles on the same chromosome tend to segregate together more often than expected by chance if their transmission was independent.

Lint Index: The weight of lint per seed in milligrams.

Lint Percent: The percentage of the seed cotton that is lint, handpicked samples.

Lint Yield: Refers to the measure of the quantity of fiber produced on a given unit of land. Presented below in pounds of lint per acre.

Lint/boll: As used herein, the term “lint/boll” is the weight of lint per boll.

Maturity Rating: A visual rating near harvest on the amount of open boils on the plant. The rating range is from 1 to 5, 1 being early and 5 being late.

Micronaire (Mic): Refers to a measure of fiber fineness (high=coarse fiber) as measured with an HVI machine. Within a cotton cultivar, micronaire is also a measure of maturity. Micronaire differences are governed by changes in perimeter or in cell wall thickness, or by changes in both. Within a variety, cotton perimeter is fairly consistent and maturity will cause a change in micronaire. Consequently, micronaire has a high correlation with maturity within a variety of cotton. Maturity is the degree of development of cell wall thickness.

Mr: Fiber maturity ratio.

Phenotype: The detectable characteristics of a cell or organism, which characteristics are the manifestation of gene expression.

Plant Height: The average height in meters of a group of plants.

Quantitative Trait Loci (QTL): Quantitative trait loci (QTL) refer to genetic loci that control to some degree numerically representable traits that are usually continuously distributed.

Recurrent Parent: The repeating parent (variety) in a backcross breeding program. The recurrent parent is the variety into which a gene or trait is desired to be introduced.

Regeneration: The development of a plant from tissue culture.

Seed/boll: Refers to the number of seeds per boll, handpicked samples.

Seedcotton/boll: Refers to the weight of seedcotton per boll, handpicked samples.

Seedweight: Refers to the weight of 100 seeds in grams.

Self-pollination: The transfer of pollen from the anther to the stigma of the same plant or a plant of the same genotype.

Single Locus Converted (Conversion) Plant: Plants which are developed by a plant breeding technique called backcrossing wherein essentially all of the desired morphological and physiological characteristics of a variety are recovered in addition to the characteristics conferred by the single locus transferred into the variety via the backcrossing technique. A single locus may comprise one gene, or in the case of transgenic plants, one or more transgenes integrated into the host genome at a single site (locus).

Stringout Rating: also referred to as “Storm Resistance”, refers to a visual rating prior to harvest of the relative looseness of the seed cotton held in the boll structure on the plant. The rating values are from 1 to 5 (tight to loose in the boll).

Substantially Equivalent: A characteristic that, when compared, does not show a statistically significant difference (e.g., p=0.05) from the mean.

T1: A measure of fiber strength, grams per tex (high=stronger fiber).

Tissue Culture: A composition comprising isolated cells of the same or a different type or a collection of such cells organized into parts of a plant.

Transgene: A genetic locus comprising a sequence which has been introduced into the genome of a cotton plant by transformation.

Twinlink: a breeding stack of the man-made cotton locus containing the cry2Ae gene and further described in APHIS petition No. 08-340-01p (referred to as GHB119) in combination with the man-made cotton locus containing the cry1 Ab gene and further described in APHIS petition No. 08-340-01p (referred to as T304-40).

Twinlink Plus: a breeding stack of GlyTo1 together with Twinlink and COT102.

Uniformity Ratio (Ur): The proportion of uniform length fibers. The uniformity ratio is determined by dividing the 50% fiber span length by the 2.5% fiber span length.

Vegetative Nodes: The number of nodes from the cotyledonary node to the first fruiting branch on the main stem of the plant.

### Deposit

A deposit of the BASF Agricultural Solutions Seed US LLC proprietary cotton variety FM 823AXTP disclosed above and recited in the appended claims is maintained by BASF Agricultural Solutions Seeds US LLC. A deposit under the Budapest Treaty will be made with the Provasoli-Guillard National Center for Marine Algae and Microbiota, Bigelow Laboratory for Ocean Sciences (NCMA), 60 Bigelow Drive, East Boothbay, Maine 04544. Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period. Upon allowance of any claims in the application, the Applicant will make available to the public, pursuant to 37 C.F.R. § 1.808, sample(s) of the deposit.

1. A seed of cotton variety FM 823AXTP, wherein representative seed of said variety was deposited under NCMA No.______.

2. A plant, or a regenerable part thereof, produced by growing the seed of claim 1.

3. A plant, or a regenerable part thereof, obtained by vegetative reproduction from the plant, or a part thereof, of claim 2, wherein said plant expresses all of the morphological and physiological characteristics of cotton variety FM 823AXTP, a sample of seed of cotton variety FM 823AXTP having been deposited under NCMA No.______.

4. A process of vegetative reproduction of cotton variety FM 823 AXTP comprising culturing regenerable cells or tissue from FM 823 AXTP, a sample of seed of cotton variety FM 823 AXTP having been deposited under No.______.

5. A cell or tissue culture produced from the plant, or a part thereof, of claim 2.

6. A cotton plant regenerated from the cell or tissue culture of claim 5, said plant expressing all of the morphological and physiological characteristics of FM 823AXTP, a sample of seed of cotton variety FM 823AXTP having been deposited under NCMA No.______.

7. A method of producing an F₁ hybrid cotton seed, comprising the steps of crossing the plant of claim 2 with a different cotton plant and harvesting the resultant F₁ hybrid cotton seed.

8. An F₁ hybrid cotton seed produced by the method of claim 7.

9. An F₁ hybrid cotton plant, or a regenerable part thereof, produced by growing the hybrid seed of claim 8.

10. A method of introducing a desired trait into a cotton plant, the method comprising transforming the plant of claim 2 with a transgene that confers the desired trait, wherein the transformed plant otherwise retains all of the morphological and physiological characteristics of cotton variety FM 823AXTP and contains the desired trait.

11. The method of claim 10, wherein said desired trait is fiber quality, herbicide resistance, insect resistance, bacterial disease resistance or fungal disease resistance.

12. A method of introducing a desired trait into a cotton plant, the method comprising transforming the plant of claim 9 with a transgene that confers the desired trait, wherein the transformed plant otherwise retains all of the morphological and physiological characteristics of cotton variety FM 823AXTP and contains the desired trait, seed of cotton variety FM 823 AXTP having been deposited under NCMA No.______.

13. A cotton plant produced by the method of claim 10.

14. A method of introducing a single locus conversion into cotton variety FM 823AXTP comprising: (a) crossing a plant of variety FM 823 AXTP with a second plant comprising a desired single locus to produce F₁ progeny plants; (b) selecting F₁ progeny plants that have the single locus to produce selected F₁ progeny plants; (c) crossing the selected progeny plants with at least a first plant of variety FM 823AXTP to produce backcross progeny plants; (d) selecting backcross progeny plants that have the single locus and otherwise comprise all of the physiological and morphological characteristics of cotton variety FM 823 AXTP to produce selected backcross progeny plants; and (e) repeating steps (c) and (d) one or more times in succession to produce selected second or higher backcross progeny plants that comprise the single locus and otherwise comprise all of the physiological and morphological characteristics of cotton variety FM 823AXTP when grown in the same environmental conditions, wherein a representative seed of said variety has been deposited under NCMA No.______.

15. The method of claim 14, wherein the single locus confers male sterility, herbicide tolerance, insect resistance, pest resistance, disease resistance, modified fatty acid metabolism, modified carbohydrate metabolism, or modified cotton fiber characteristics.

16. A method of producing a commodity plant product, wherein the method comprises collecting the commodity plant product from the plant of claim 2.

17. A commodity plant product that is produced by the method of claim 16, wherein the commodity plant product comprises at least one cell of cotton variety FM 823AXTP.
